(19) United States
(12) Patent Application Publication
(10) Pub. No.: US 2002/0026945 A1
(43) Pub. Date: Mar. 7, 2002

(54) Enhancement of photodynamic therapy by anti-angiogenic treatment

(76) Inventors: Charles J. Gomer, Glendora, CA (US); Parkash S. Gill, Agoura, CA (US)

Correspondence Address: Benjamin Aaron Adler, ADLER & ASSOCIATES, 8011 Candle Lane, Houston, TX, 77071, US

(21) Appl. No.: 09/912,756

(22) Filed: Jul. 24, 2001

Related U.S. Application Data
(60) Provisional application No. 60/220,311, filed on Jul. 24, 2000.

(51) Int. Cl. A61B 19/00

(52) U.S. Cl. 128/898

(57) ABSTRACT

Photodynamic therapy mediated oxidative stress elicits both direct tumor cell damage as well as microvascular injury within exposed tumors. Reduction in vascular perfusion associated with PDT mediated microvascular injury produces tumor tissue hypoxia. In a transplantable BA mouse mammary carcinoma, Photofrin mediated PDT induced expression of the hypoxia inducible factor-1 alpha (HIF-1α) subunit of the heterodimeric HIF-1 transcription factor and also increased protein levels of the HIF-1 target gene, vascular endothelial growth factor, within treated tumors. Tumor bearing mice treated with combined anti-angiogenic therapy (IM862 or EMAP-II) and PDT had improved tumoricidal responses compared to individual treatments. PDT induced VEGF expression in tumors decreased when either IM862 or EMAP-II was included in the PDT treatment protocol. Combination procedures using anti-angiogenic treatments improves the therapeutic effectiveness of PDT.

### CROSS-REFERENCE TO RELATED APPLICATIONS

This non-provisional application claims benefit of priority of provisional patent application U.S. Ser. No. 60/220,311, filed Jul. 24, 2000, now abandoned.

### FEDERAL FUNDING LEGEND

This invention was produced in part using funds from the Federal government under USPHS grant Nos. CA-31230, HL-60061, and HL-03981 from the National Institutes of Health and Office of Naval Research grant N000014-91-J-4047 and U.S. Army Medical Research grant BC981102 from the Department of Defense. Accordingly, the Federal government has certain rights in this invention.

### BACKGROUND OF THE INVENTION

1. Field of the Invention

The present invention relates generally to the clinical treatment of solid tumors. More specifically, the present invention relates to anti-angiogenic treatment for the enhancement of photodynamic therapy.

2. Description of the Related Art

Photodynamic therapy (PDT) involves treating solid malignancies with tissue penetrating laser light following the systemic administration of a tumor localizing photosensitizer (1). Properties of photosensitizer localization in tumor tissue and photochemical generation of reactive oxygen species are combined with precise delivery of laser generated light to produce a treatment offering local tumoricidal activity (2,3). The porphyrin photosensitizer, Photofrin (PH), recently received FDA approval for photodynamic therapy treatment of esophageal and endobronchial carcinomas (1). Photodynamic therapy is also undergoing clinical evaluation for the treatment of bladder, head & neck, brain, intrathoracic, and skin malignancies (1). Photodynamic therapy targets include tumor cells, tumor microvasculature, inflammatory cells, and immune host cells (1-3).

Vascular effects induced by PH-mediated photodynamic therapy include perfusion changes, vessel constriction, macromolecular vessel leakage, leukocyte adhesion and thrombus formation (1,4). These effects appear to be linked to platelet activation and release of thromboxane (5). Microvasculature damage is readily observed histologically following photodynamic therapy and leads to a significant decrease in blood flow as well as severe and persistent tumor tissue hypoxia (6,7). Rapid and substantial reductions in tissue oxygenation can also occur during illumination by direct utilization of oxygen during the photochemical generation of reactive oxygen species (7,8).

Tissue hypoxia induces a plethora of molecular and physiological responses including an adaptive response associated with gene activation (9). A primary step in hypoxia mediated gene activation is the formation of the HIF-1 transcription factor complex (9,10). HIF-1 is a heterodimeric complex of two helix-loop-helix proteins, HIF-1β (ARNT) and HIF-1α (11). ARNT is constitutively expressed while HIF-1α is rapidly degraded under normoxic conditions. Hypoxia induces the stabilization of the HIF-1α subunit which in turn allows for the formation of the transcriptionally active protein complex (11,12). A number of HIF-1 responsive genes have been identified including VEGF, erythropoietin, and glucose transporter-1 (11). VEGF, also called vascular permeability factor, is an endothelial cell specific mitogen involved with the induction and maintenance of the neovasculature in solid tumors (11,13). VEGF expression increases in tumor tissue under hypoxia as a result of both transcriptional activation and increased stabilization (11,14).

Photodynamic therapy continues to show promise in the treatment of a variety of malignant and non-malignant disorders (1,19). The use of photodynamic therapy for advanced esophageal tumors offers prolonged tumor responses when compared to standard Nd-YAG laser ablation treatments. Extended tumor responses are also observed in advanced non-small cell lung cancer patients treated with photodynamic therapy as compared to Nd-YAG laser ablation. Likewise, photodynamic therapy applications continue to be encouraging for early stage lung cancer, brain cancers, head & neck cancers, and for non-oncologic disorders such as age related macular degeneration (1,19). Nevertheless recurrences are observed following photodynamic therapy and methods to improve the therapeutic efficacy of this procedure are needed. Multiple physiological, biophysical, and/or pharmacological variables may account for recurrences following photodynamic therapy (2,3). Non-uniform distribution of photosensitizers within tumor tissue, inadequate light distribution, photosensitizer photobleaching, and treatment induced oxygen deprivation may all contribute to suboptimal photodynamic therapy responses.

The prior art is deficient in the lack of methods to improve the therapeutic efficacy of photodynamic therapy. The present invention fulfills this longstanding need and desire in the art.

### SUMMARY OF THE INVENTION

Other and further aspects, features, and advantages of the present invention will be apparent from the following description of the presently preferred embodiments of the invention. These embodiments are given for the purpose of disclosure.

The instant invention resulted from attempts to determine whether photodynamic therapy-induced microvascular damage and resulting hypoxia could serve as an activator of molecular events leading to the increased expression of VEGF within treated tumor tissue. As it was determined that Photofrin porfimer sodium (PH) mediated PDT induces expression of HIF-1α and the transcription factor's target gene VEGF in a transplanted mouse mammary carcinoma, it was hypothesized that anti-angiogenic compounds, which counter the actions of VEGF, could improve photodynamic therapy tumor responsiveness. The Examples herein demonstrate that enhanced tumoricidal activity results when photodynamic therapy is combined with anti-angiogenic therapy.

Possible photosensitizers for photodynamic therapy therapy include Photofrin, tin etiopurpurin (SnET2), mono-1-aspartyl chlorin e6 (NPe6), benzoporphyrin derivative (BPD), meso-tetra- (hydroxyphenyl) chlorin (mTHPC) and 5-amino levulinic acid (ALA). The examples of the instant invention utilize Photofrin porfimer sodium, which is activated by 630 nm red light irradiation from a non-thermal laser. Representative anti-angiogenic agents include inhibitors of VEGF expression such as IM862 or EMAP-II. The anti-angiogenic agent may be administered locally or systemically. It is expected that the instant invention should be useful in the treatment of tumors and other areas of abnormal tissue or blood vessel growth.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A and 1B show that photodynamic therapy treatment of BA mammary carcinoma tumors growing in C3H mice induced expression of the transcription factor subunit HIF-1α and VEGF. In FIG. 1A, tumors were collected immediately following treatment and evaluated for HIF-1α expression by Western immunoblot analysis. HIF-1α was not detectable in control tumors measuring 6-7 mm in diameter. Both photodynamic therapy (PH, 5 mg/kg; 200 J/cm²) and tumor clamping (45 min) induced HIF-1α expression. In FIG. 1B, separate tumors were collected 24 hr after photodynamic therapy or clamping (45 min) and assayed for VEGF expression by Western immunoblot analysis. Expression of actin was used to monitor protein loading.

FIG. 2 shows VEGF levels in culture media from control BA mammary carcinoma cells and from cells exposed to light alone, CoCl₂, PH alone or photodynamic therapy. Culture media was collected 2, 6 or 24 hr after treatment and VEGF concentrations were determined by ELISA. Each group represents the mean ± SE of 5 individual experiments. A statistically significant difference in VEGF levels was observed only between CoCl₂ and control samples (p<0.05).

FIG. 3 demonstrates that anti-angiogenic treatments enhance the tumoricidal action of photodynamic therapy. C3H mice transplanted with BA mammary carcinomas received daily injections for 10 days of either IM-862 (25 mg/kg per dose, n=9) or EMAP-II (50 μg/kg per dose, n=9) commencing 1 hr prior to a single PDT treatment (5 mg/kg PH, 200 J/cm²). Mice were monitored for tumor recurrences 3 times per week for 40 days. Control conditions included individual anti-angiogenic treatments alone (n=9) and photodynamic therapy treatment alone (n=18). There was a statistically significant difference in percent cures between photodynamic therapy alone versus photodynamic therapy +EMAP-II or PDT+IM862 (p<0.05).

FIG. 4 demonstrates that anti-angiogenic compounds IM-862 and EMAP-II can decrease VEGF levels in photodynamic therapy treated tumors. Tumor bearing mice received either no treatment (Control), photodynamic therapy alone, or photodynamic therapy plus 2 injections of either EMAP-II or IM862 (1 hr prior to photodynamic therapy and 23 hr after photodynamic therapy). Tumor samples were collected 24 hr after PDT and assayed for VEGF expression using a commercial ELISA assay kit. Each group represents the mean ± SE of 6 individual tumor samples. There was a statistically significant difference in VEGF levels between photodynamic therapy alone and photodynamic therapy plus EMAP-II (p<0.01).

### DETAILED DESCRIPTION OF THE INVENTION

As appearing herein, the following terms shall have the definitions set out below.

As used herein, the term “photodynamic therapy” or “PDT” refers to the treatment of solid tumors with visible light (usually generated by non-thermal lasers) following the systemic administration of a tumor localizing photosensitizer (see Fisher, A. M. R., et al., Laser Surgery Medicine 17:2-31 (1995); Marcus, S. L. and Dugan, M. H., Laser Surgery Medicine, 12: 318-24 (1992); and Henderson, B. W. and Dougherty, T. J., Photochem. Photobiol., 55:931-48 (1992)). The photochemical reaction induced by the photosensitizer and laser light produces reactive oxygen species such as singlet oxygen, which in turn induces oxidative damage to subcellular targets (membranes, organelles, enzymes, and DNA). PDT is used clinically to treat various types of solid tumors (esophagus, bronchus, bladder, brain, eye, head/neck, skin, cervical as well as non-malignant diseases such as age related macular degeneration and psoriasis. Various photosensitizers, including Photofrin (PH), tin etiopurpurin (SnET2), mono-1-aspartyl chlorin e6 (NPe6), benzoporphyrin derivative (BPD), meso-tetra-(hydroxyphenyl) chlorin (mTHPC) and 5-amino levulinic acid (ALA) are used in photodynamic therapy.

As used herein, the term “anti-angiogenic agent” and “anti-angiogenic treatment” refers to agents or treatments respectively which reduce or terminate the formation of blood vessels.

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual” (1982); “DNA Cloning: A Practical Approach,” Volumes I and II (D. N. Glover ed. 1985); “Oligonucleotide Synthesis” (M. J. Gait ed. 1984); “Nucleic Acid Hybridization” (B. D. Hames & S. J. Higgins Eds. (1985)); “Transcription and Translation” (B. D. Hames & S. J. Higgins Eds. (1984)); “Animal Cell Culture” (R. I. Freshney, ed. (1986)); “Immobilized Cells And Enzymes” (IRL Press, (1986)); B. Perbal, “A Practical Guide To Molecular Cloning” (1984)).

Abbreviations used herein are: ARNT, aryl hydrocarbon nuclear receptor-translocator; BSA, bovine serum albumin; EMAP-II, endothelial-monocyte activating polypeptide; FCS, fetal calf serum; HIF-1, hypoxia inducible transcription factor; HRE, hypoxia response element; PDT, photodynamic therapy; PH, Photofrin porfimer sodium; TNF, tumor necrosis factor; and, VEGF, vascular endothelial growth factor.

The current invention is directed to a method of increasing therapeutic efficacy of photodynamic therapy in a target tissue by combining photodynamic therapy with administration of an antiangiogenic agent. Representative photosensitizers include Photofrin, tin etiopurpurin (SnET2), mono-1-aspartyl chlorin e6 (NPe6), benzoporphyrin derivative (BPD), meso-tetra-(hydroxyphenyl) chlorin (mTHPC) and 5-amino levulinic acid (ALA). Preferably, the photosensitizer is Photofrin porfimer sodium, which is activated by 630 nm red light irradiation from a non-thermal laser. Light of this wavelength can be produced by an argon pumped dye laser.

Representative anti-angiogenic agents include inhibitors of VEGF expression such as IM862. EMAP-II is another anti-angiogenic agent useful in the instant invention. The anti-angiogenic agent may be administered locally or systemically. The target tissue may be a tumor, an area of abnormal tissue growth, or an area of abnormal blood vessel growth. Specific example may include mammary carcinomas, an esophageal carcinomas, endobronchial carcinomas, bladder tumors, cervical tumors, head & neck tumors, brain tumors, intrathoracic tumors, lung tumors, skin malignancies, age related macular degeneration and psoriasis.

The following examples are given for the purpose of illustrating various embodiments of the invention and are not meant to limit the present invention in any fashion.

### EXAMPLE 1

Drugs and Reagents

The photosensitizer Photofrin porfimer sodium was a gift from QLT PhotoTherapeutics, Inc. (Vancouver, British Columbia, Canada) and was dissolved in 5% dextrose in water to make a 2.5 mg/ml stock solution. Recombinant EMAP-II was prepared as previously described (15). A working solution at 10 μg/ml was prepared in PBS containing 0.1% BSA. IM862 was obtained from Cytran Inc. (Kirkland, Wash.) and was dissolved in saline to make a 5 mg/ml working solution (16). CoCl₂ was obtained from Sigma Chemical Co., St. Louis, Mo. and a 10 mM stock solution was prepared in water.

### EXAMPLE 2

Cells and In-Vivo Tumor Model.

BA mouse mammary carcinoma cells (originally obtained from the NIH tumor bank) were used in all in-vitro and in-vivo experiments (17). Cells were grown as a monolayer in RPMI 1640 media supplemented with 10% fetal calf serum (FCS) and antibiotics. The plating efficiency for the BA cells ranged from 40-60%. Subcutaneous BA mammary carcinomas were generated by trocar injection of 1-mm³ pieces of tumor to the hind right flank of 8-12 week old female C3H/HeJ mice (17).

### EXAMPLE 3

In-vitro and In-vivo Treatment Protocols.

In-vitro photosensitization protocols involved seeding cells into plastic Petri dishes and incubating overnight in complete growth media to allow for cell attachment. PDT treatments included incubating cells in the dark at 37° C. for 16 h with PH (25 μg/ml) in media containing 5% FCS. Cells were then incubated for an additional 30 min in growth media containing 10% FCS, rinsed in media without serum, and exposed to red light (570-650 nm) generated by a parallel series of red milar filtered 30 watt fluorescent bulbs and delivered at a dose rate of 0.35 mW/cm². In specified experiments, cells were incubated with CoCl₂ (100 μM) in growth media containing 5% FCS for 16 hr. Treated cells were then refed with complete growth media and incubated in the dark at 37° C. until collected for analysis of VEGF secretion into the culture media.

In-vivo photodynamic therapy tumor treatments were performed as previously reported on tumors measuring 6-7 mm in diameter (17). Briefly, photodynamic therapy procedures included an i.v. injection of PH (5 mg/kg) followed 24 h later with non-thermal laser tumor irradiation using an argon pumped dye laser emitting red light at 630 nm. A light dose rate of 75 mW/cm² and a total light dose of 200 J/cm² were used for all in-vivo PDT treatments. Following treatment, tumors were measured 3 times per week. Cures were defined as being disease free for at least 40 days following photodynamic therapy (17). Anti-angiogenic treatment was performed using either EMAP-II or IM862. Each compound was administered as daily IP injections for 10 consecutive days starting 1 h prior to photodynamic therapy light treatment. Individual IM862 doses were 25 mg/kg and individual EMAP-II doses were 50 μg/kg. Tumor tissue hypoxia was induced in selected experiments by clamping lesions for 45 minutes. Statistical analysis was performed using the X² test for evaluation of tumor cure rates.

### EXAMPLE 4

Western Blot Analysis

Tumors were collected at various times after treatment, homogenized with a polytron in 1X reporter lysis buffer (Promega, Wis.) and evaluated for protein expression as described previously (18). Briefly, protein samples (30 μg) were size separated on 10% (for HIF-1α) or 12.5% (for VEGF) discontinuous polyacrylamide gels and transferred overnight to nitrocellulose membranes. Filters were blocked for 1 h with 5% nonfat milk and then incubated for 2 h with either a mouse monoclonal anti-HIF-1α antibody (Clone 54, Transduction Laboratories, Lexington, Ky.), a rabbit polyclonal anti-VEGF antibody (No. sc-507, Santa Cruz Biotechnology, Santa Cruz, Calif.) or a mouse monoclonal anti-actin antibody (clone C-4, ICN, Aurora, Ohio). Filters were then incubated with either an anti-mouse or anti-rabbit peroxidase conjugate (Sigma, St. Louis, Mo.) and the resulting complexes visualized by enhanced chemiluminescence autoradiography (Amersham Life Science, Chicago, Ill.).

### EXAMPLE 5

ELISA Assays.

A Quantikine M mouse VEGF ELISA kit (R&D Systems, Minneapolis, Minn.) was used to quantify VEGF levels in cell culture media as well as in tumor extracts from control and treated mice. Results were normalized to protein concentrations from tumor tissue or cell lysates. Statistical analysis was performed using a 2-tailed Student's t test to analyze VEGF levels.

### EXAMPLE 6

Photodynamic Therapy and Hypoxia.

Molecular events associated with photodynamic therapy induced hypoxia were analyzed with an emphasis on determining whether photodynamic therapy effectiveness were enhanced with anti-angiogenic therapy. Several laboratories have shown that photodynamic therapy produces microvascular damage within treated tumors and that photodynamic therapy leads to tumor tissue hypoxia (4-8). Hypoxia mediates adaptive gene expression through the HIF transcription factor (9). An initial step in hypoxia mediated gene activation is the formation of the HIF-1 heterodimeric transcription factor complex (10). One subunit, HIF-1 (ARNT), is constitutively expressed while the second subunit, HIF-1α is rapidly degraded under normoxic conditions by the ubiquitin-proteosome system (9,10,12). Since hypoxia induces increased expression and stabilization of the HIF-1α subunit as well as activates the HIF-1 transcription complex, it seemed likely that photodynamic therapy induced microvascular damage and resulting tumor tissue hypoxia could also stabilize HIF-1α and initiate HIF-1 mediated transcription.

### EXAMPLE 7

Effects of Photodynamic Therapy on HIF-1α Expression

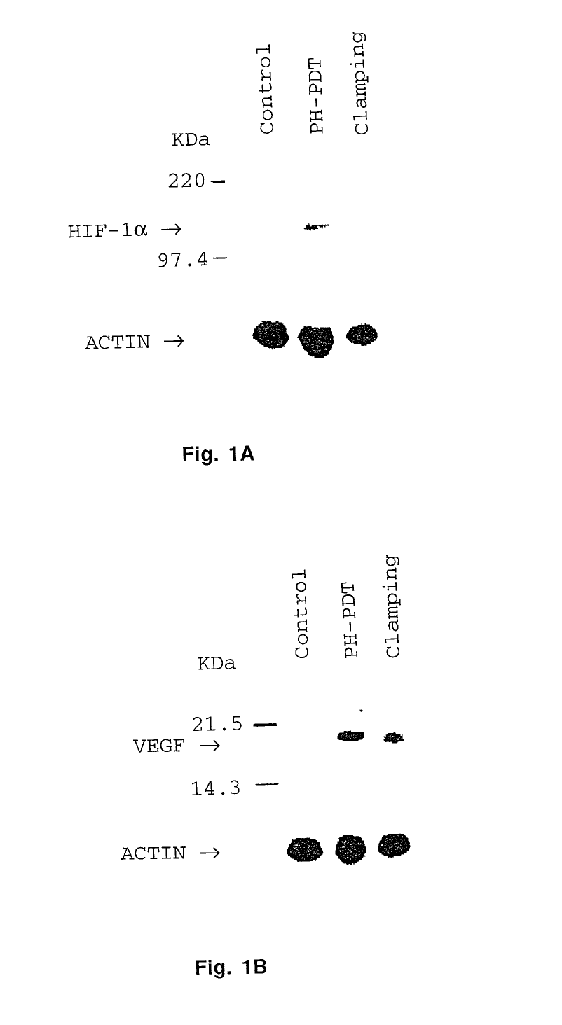

FIG. 1A shows western analysis indicating that photodynamic therapy treatment of BA mammary carcinoma tumors growing in C3H mice induced expression of HIF-1α. This response was rapid, being observed within the first 5 minutes following photodynamic therapy. Tumor clamping was used as a positive control and resulted in comparable HIF-1α expression. The HIF-1α complex functions via binding to an HRE found in the promoter region of the VEGF gene as well as in the 3′ flanking region of the erythropoietin gene (11). Expression of VEGF in areas around histologically documented tumor necrosis originally led to suggestions that hypoxia is a major regulator of tumor angiogenesis (13,14).

### EXAMPLE 8

Effects of Photodynamic Therapy on VEGF Expression

VEGF is a dimeric glycoprotein with strong mitogenic activity restricted primarily to endothelial cells (14). FIG. 1B documents VEGF expression following in-vivo photodynamic therapy. Western analysis was performed under reducing conditions on tumor lysates collected 24 hours after photodynamic therapy. Photodynamic therapy and tumor clamping both induced significant increases in VEGF expression within treated lesions. VEGF induced angiogenesis plays an important role in tumor growth. Inhibition of VEGF activity with neutralizing antibodies inhibits the growth of primary and metastatic tumors, and attenuation of VEGF expression decreases tumor growth and vascularity (20). These results suggest that photodynamic therapy may be functioning as a mediator of tumor angiogenesis and tumor recurrence by enhancing expression of VEGF within the treated tumor mass (14).

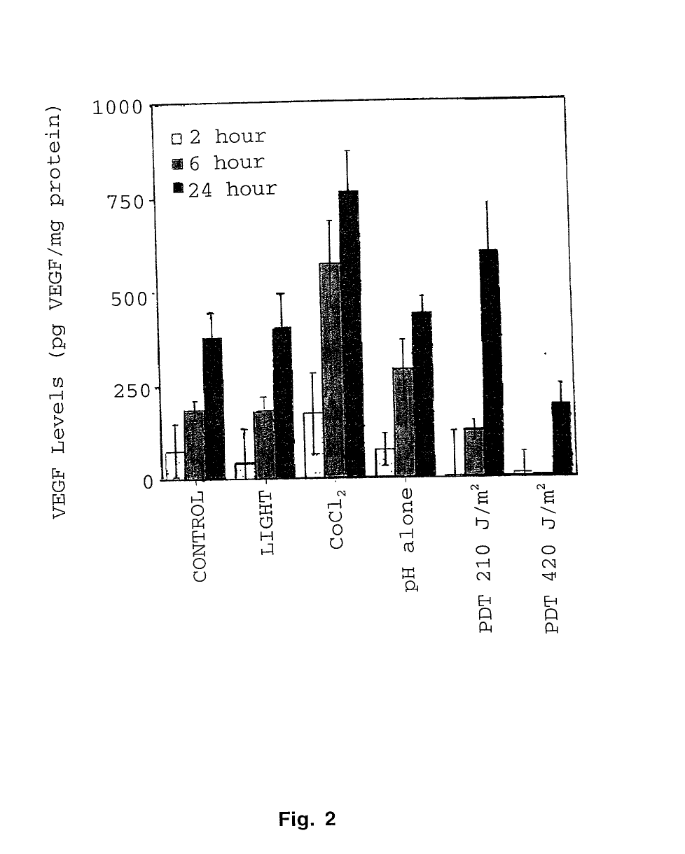

In-vitro photodynamic therapy on BA mammary carcinoma also induced expression of VEGF. FIG. 2 shows VEGF levels collected from culture media at various time intervals for control and treatment conditions. Exposure to CoCl₂ served as a statistically significant positive control since exposure to this divalent metal induces cellular VEGF expression (11). A 210 J/m² photodynamic therapy dose resulted in a modestly increase in VEGF levels when measured 24 hr after treatment. The photodynamic therapy doses (210 and 420 J/m²) and CoCl₂ treatment produced clonogenic survival levels ranging from 33% to 96%. The in-vitro PDT conditions would be expected to involve singlet oxygen mediated oxidative stress but not induced hypoxia.

These results suggest that the increase in VEGF expression observed in tumors following in-vivo photodynamic therapy may be associated with treatment induced hypoxia and to a lesser extent with treatment induced oxidative stress. Exposure of various mouse and human tumor cells to ionizing radiation and exposure of rat endothelial cells to hydrogen peroxide can upregulate VEGF expression (20,21). Additional studies can determine similarities and differences in VEGF induction for various types of oxidative stress.

### EXAMPLE 9

Anti-angiogenic Agents Enhance Photodynamic Therapy

A growing number of reports indicate that anti-angiogenic agents can enhance the tumoricidal effectiveness of chemotherapy and radiation treatments (20,22,23). Anti-angiogenic treatments using either EMAP-II or IM862 were examined to determine whether these treatments could enhance the tumoricidal action of PDT.

EMAP-II is a single chain polypeptide which inhibits tumor growth and has anti-angiogenic activity (15). EMAP-II induces apoptosis in growing capillary endothelial cells in both a time and dose dependent manner. EMAP-II also prevents vessel ingrowth in experimental angiogenesis models and in primary tumors. Interestingly, EMAP-II does not induce toxicity in normal organs.

IM862 is a dipeptide of L-glutamyl-L-tryptophan that was initially isolated from the thymus (16). Preclinical studies have shown that the dipeptide inhibits angiogenesis in chorioallantoic membrane assays and inhibits VEGF production in monocytic lineage cells. IM862 also inhibits tumor growth in xenograft models but has no direct cytotoxic effect on tumor cells. IM862 mediates these effects by inhibiting production of VEGF and by activating natural killer cells. Intranasal administration of IM862 exhibits antitumor activity in patients with AIDS associated Kaposi's sarcoma (16). IM862 also appears to be safe and well tolerated when delivered over prolonged time periods.

A photodynamic therapy procedure, which produced a moderate cure rate alone, was used to measure positive or negative changes in tumor response when a single photodynamic therapy treatment was combined with daily injections of EMAP-II or IM862 for 10 days (17).

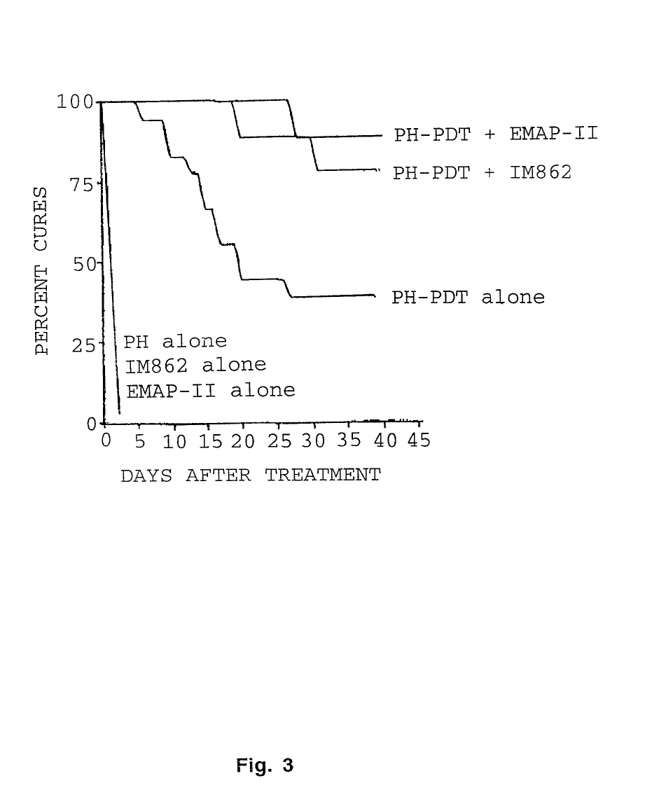

FIG. 3 shows that anti-angiogenic treatment statistically enhanced (p<0.05) the tumoricidal action of photodynamic therapy as measured by tumor cures. Specifically, the 200 J/cm² photodynamic therapy dose alone produced a 39% cure rate while photodynamic therapy plus EMAP-II or IM862 produced tumor cures of 89% and 78% respectively. The anti-angiogenic treatments alone did not produce any tumor cures or tumor regression and only slightly modified tumor growth parameters.

### EXAMPLE 10

Effects of EMAP-11 and IM862 on PDT Induction of VEGF Expression

The effects of the anti-angiogenic derivatives on photodynamic therapy induced VEGF levels were analyzed in the treated tumors. The in-vivo photodynamic therapy dose delivered to tumors (200 J/cm²) induced rapid and severe tissue necrosis. Therefore, tumor samples were only collected 24 hr following photodynamic therapy. This time frame allowed for two anti-angiogenic drug doses (1 hr prior to light treatment and 1 hr prior to sacrifice).

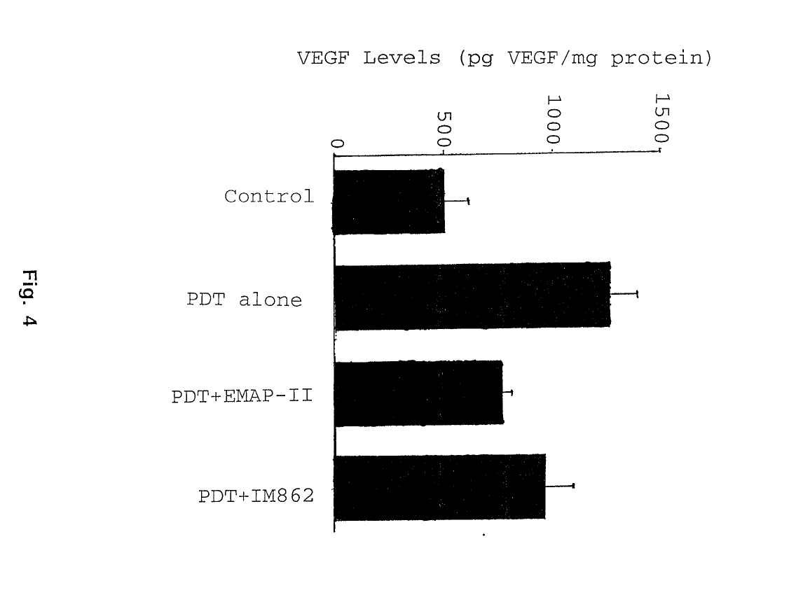

FIG. 4 shows a decrease in VEGF levels, measured by ELISA, when photodynamic therapy was combined with EMAP-II or IM862 compared to photodynamic therapy treated tumors alone. These results were obtained after only 2 doses of either EMAP-II or IM862. Nevertheless, a statistically significant decrease (p<0.01) in photodynamic therapy induced VEGF levels was observed when EMAP-II was included in the treatment protocol. It is likely the 10 daily doses of EMAP-II or IM862 used in the photodynamic therapy tumor treatment experiments would further attenuate VEGF levels.

Summary

The results presented herein demonstrate that antiangiogenic treatments can potentiate photodynamic therapy responsiveness. This result may involve attenuating the angiogenic actions of VEGF, which was observed to increase in photodynamic therapy treated tumors. The minimal systemic toxicity associated with anti-angiogenic therapy indicates that these procedures are compatible with clinical photodynamic therapy and provide an efficient strategy for selectively enhancing photodynamic therapy tumor responsiveness.

The following references were cited herein:

1. Dougherty, et al., Photodynamic Therapy. J. Natl. Cancer Inst., 90: 889-905, 1998.

2. Henderson, B. W. and Dougherty, T. J. How does photodynamic therapy work? Photochem. Photobiol., 55: 145-157, 1992.

3. Oleinick, N. L. and Evans, H. E. The photobiology of photodynamic therapy: cellular targets and mechanisms. Radiat. Res., 150: S146-156, 1998.

4. Fingar, et al., The role of microvascular damage in photodynamic therapy: the effect of treatment on vessel constriction, permeability, and leukocyte adhesion. Cancer Res. 52: 4914-4921, 1992.

5. Fingar, V. H., Wieman, T. J., and Haydon, P. S. The effects of thrombocytopenia on vessel stasis and micromolecular leakage after photodynamic therapy using Photofrin. Photochem. Photobiol., 66: 513-517, 1997.

6. van Geel, et al., Vascular perfusion and hypoxic areas in RIF-1 tumours after photodynamic therapy. Br. J. Cancer, 73: 288-293, 1996.

7. Sitnik, T. M., Hampton, J. A. and Henderson, B. W. Reduction of tumor oxygenation during and after photodynamic therapy in-vivo: Effects of fluence rate. Br. J. Cancer, 77: 1386-1394, 1998.

8. Foster, et al., Oxygen consumption and diffusion effects in photodynamic therapy. Radiat. Res., 126: 296-303, 1991.

9. Ratcliffe, et al., Oxygen sensing, hypoxia-inducible factor-1 and the regulation of mammalian gene expression. J. Exp. Biol., 201: 1153-1162, 1998.

10. Wang, G. L., and Semenza, G. L. General involvement of hypoxia-inducible factor in transcriptional response to hypoxia. Proc. Natl. Acad. Sci. USA, 90: 4303-4308, 1993.

11. Forsythe, et al., Activation of vascular endothelial growth factor gene transcription by hypoxia-inducible factor 1. Mol. Cell Biol., 16: 4604-4613, 1996.

12. Huang, L. E., Gu, J., Schau, M., and Bunn, H. F. Regulation of hypoxia-inducible factor la is mediated by an oxygen dependent degradation domain via the ubiquitin proteasome pathway. Proc. Natl. Acad. Sci. USA, 95: 7987-7992, 1998.

13. Senger, D. R., Peruzzi, C. A., Feder, J., and Dvorak, H. F. A highly conserved vascular permeability factor secreted by a variety of human and rodent tumor cell lines. Cancer Res., 46: 5629-5632, 1986.

14. Shweiki, D., Itin, A., Soffer, D., and Keshet, E. Vascular endothelial growth factor induced by hypoxia may mediate hypoxia-initiated angiogenesis. Nature (London), 359: 843-845, 1992.

15. Schwarz, et al., Endothelial-monocyte activating polypeptide II, a novel antitumor cytokine that suppresses primary and metastatic tumor growth and induces apoptosis in growing endothelial cells. J. Exp. Med., 190: 341-353, 1999.

16. Tulpule, A., Scadden, D. T., Espina, B. M., Cabriales, S., Howard, W., Shea, K., and Gill, P. S. Results of a randomized study of IM862 nasal solution in the treatment of AIDS-related Kaposi's Sarcoma. J. Clin. Onc., 18: 716-723, 2000.

17. Ferrario, A., Kessel, D. and Gomer, C. J. Metabolic properties & photosensitizing responsiveness of mono-1-aspartyl chlorin e6 in a mouse tumor model. Cancer Res., 52: 2890-2893, 1992.

18. Fisher, A. M. R., Ferrario, A., Rucker, N., Zhang, S., and Gomer, C. J. Photodynamic therapy sensitivity is not altered in human tumor cells after abrogation of p53 function. Cancer Res., 59: 331-335, 1999.

19. Miller, et al., Photodynamic therapy with verteporfin for choroidal neovascularization caused by age-related macular degeneration: results of a single treatment in a phase 1 and 2 study. Arch. Ophthalmol. 117:1161-73, 1999.

20. Gorski, et al., Blockade of the vascular endothelial growth factor stress response increases the antitumor effects of ionizing radiation. Cancer Res., 59: 3374-3378, 1999.

21. Chua, C. C., Hamdy, R. C., and Chua, B. H. Upregulation of vascular endothelial growth factor by H2O2 in rat heart endothelial cells. Free Radical Biology & Medicine, 25: 891-897, 1998.

22. Teicher, B. A., Sotomayor, E. A., and Huang, Z. D. Antiangiogenic agents potentiate cytotoxic cancer therapies against primary and metastatic disease. Cancer Res., 52: 6702-6704, 1992.

23. Mauceri, et al., Combined effects of angiostatin and ionizing radiation in antitumor therapy. Nature, 394: 287-291, 1998.

Any patents or publications mentioned in this specification are indicative of the levels of those skilled in the art to which the invention pertains. These patents and publications are herein incorporated by reference to the same extent as if each individual publication was specifically and individually indicated to b e incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of increasing therapeutic efficacy of photodynamic therapy in a target tissue comprising the step of:
combining photodynamic therapy with administration of an anti-angiogenic agent.

2. The method of claim 1, wherein said photodynamic therapy uses a photosensitizer selected from the group consisting of Photofrin, tin etiopurpurin (SnET2), mono-1-aspartyl chlorin e6 (NPe6), benzoporphyrin derivative (BPD), meso-tetra-(hydroxyphenyl) chlorin (mTHPC) and 5-amino levulinic acid (ALA).

3. The method of claim 1, wherein said photosensitizer is Photofrin porfimer sodium.

4. The method of claim 1, wherein photodynamic therapy is performed with 630 nm red light irradiation from a non-thermal laser.

5. The method of claim 1, wherein said laser is from an argon pumped dye laser.

6. The method of claim 1, wherein said anti-angiogenic agent is an inhibitor of VEGF expression.

7. The method of claim 6, wherein said anti-angiogenic agent is IM862.

8. The method of claim 6, wherein said anti-angiogenic agent is EMAP-II.

9. The method of claim 1, wherein a single administration of photodynamic therapy is followed by multiple administrations of said anti-angiogenic agent.

10. The method of claim 10, wherein said anti-angiogenic agent is administered daily.

11. The method of claim 1, wherein said anti-angiogenic agent is administered systemically.

12. The method of claim 1, wherein said anti-angiogenic agent is administered locally.

13. The method of claim 1, wherein said target tissue is selected from the group consisting of a tumor, an area of abnormal tissue growth and an area of abnormal blood vessel growth.

14. The method of claim 1, wherein said target tissue is selected from a group consisting of mammary carcinomas, an esophageal carcinomas, endobronchial carcinomas, bladder tumors, cervical tumors, head & neck tumors, brain tumors, intrathoracic tumors, lung tumors, skin malignancies, age related macular degeneration and psoriasis.
